(19) United States
(12) Patent Application Publication
(10) Pub. No.: US 2006/0127428 A1
(43) Pub. Date: Jun. 15, 2006

(54) Child recovery system using transfer-type tattoo

(76) Inventor: John Miskanic, Atlantic Beach, NY (US)

Correspondence Address: AMSTER, ROTHSTEIN & EBENSTEIN LLP, 90 PARK AVENUE, NEW YORK, NY, 10016, US

(21) Appl. No.: 11/012,590

(22) Filed: Dec. 15, 2004

(51) Int. Cl. A61K 8/18

(52) U.S. Cl. 424/401

(57) ABSTRACT

A child recovery system utilizing a transfer-type tattoo includes a laminate in series of a decal substrate material, a tattoo design (in negative) releasably disposed over the substrate material, a pressure-sensitive adhesive over and in register with the tattoo design, and a removable release sheet over the adhesive. After removal of the release sheet, the tattoo design is adapted to be transferred from the substrate material to the skin of a wearer. The tattoo design defines therewithin an open area which, after transfer to the skin of the wearer, exposes a portion of the skin of the wearer for manual imprinting with personal information regarding the wearer, and refers (in positive) a viewer thereof to the personal information imprinted within the exposed portion.

### BACKGROUND OF THE INVENTION

The present invention relates to a child recovery system which promotes the swift and safe return of a lost child, and more particularly to such a system which utilizes a transfer-type tattoo.

It will appreciated that the term “child” as used herein and in the claims encompasses an individual who is not physically a child but functions at the intelligence level of a child in certain respects (for example, an Alzheimer patient or senile adult). It will also be appreciated that the term “transfer-type tattoo” as used herein and in the claims broadly encompasses any design which can be transferred form a substrate to another surface, for example, the skin of a child.

A wide variety of systems have been employed to recover a lost or missing child within a large public area. These include relatively expensive electronic systems which sound an alarm (either an alarm carried by the child, an alarm carried by the adult, or both) whenever the distance between the adult and the child exceeds a predetermined limit. In addition to the expense of these systems, the child may object to having to carry its electronic component of the system. Further, even when a stranger finds a child carrying the child-carried component and the alarm is sounding, the stranger has no way of easily and swiftly contacting the appropriate adult (e.g., parent) to inform the adult that the lost child has been found and is available for pick-up.

While there are relatively inexpensive physical systems, these too are subject to various disadvantages. For example, an upright flag-topped pole attached to the child's body for easy identification by the adult in a crowd is no longer useful when the child has fallen, gone down stairs or managed to remove the pole and either discard or use it for dueling purposes (either as a weapon of offense or defense).

In an age where cellular and telecommunication devices—such as cell phones and/or beepers—are carried by a vast majority of adults, it would be ideal if a child could be relied upon to provide an adult stranger who finds a lost child with the cell or beeper number of the device carried by the parent or other responsible adult. Unfortunately, children of a certain age cannot be relied upon to remember the cell or beeper number and any document on which the number is recorded will either tend to become lost or simply not be drawn to the attention of the stranger who finds the lost child.

Accordingly, it is an object of the present invention to provide an inexpensive and reliable child recovery system wherein the child-carried component cannot be lost or removed by the child.

Another object is to provide such a system wherein the child-carried component provides the stranger who finds the child with the necessary information to swiftly and easily reunite the child with the adult who lost it.

A further object is to provide such a system wherein a child will enjoy bearing the child-carried component and may even draw a stranger's attention thereto.

It is also an object of the present invention to provide such a system which is simple, inexpensive and easy to manufacture and use.

### SUMMARY OF THE INVENTION

It has now been found that the above and related objects of the present invention are obtained by a transfer-type tattoo which is adapted to be transferred to a wearer. The transferred tattoo design has an open area for manual imprinting with personal information regarding the wearer and refers a viewer thereof to the personal information.

According to a preferred embodiment of the invention, a transfer- type tattoo used in a child recovery system comprises a laminate in series of a decal substrate material, a tattoo design (in negative) releasably disposed over the substrate material, a pressure-sensitive adhesive over and in register with the tattoo design, and a removable release sheet over the adhesive. After removal of the release sheet, the tattoo design is adapted to be transferred from the substrate material to the skin of a wearer. The tattoo design defines therewithin an open area which, after transfer to the skin of the wearer, exposes a portion of the skin of the wearer for manual imprinting with personal information regarding the wearer, and (in positive) refers a viewer thereof to the personal information imprinted within the open area.

In one preferred embodiment the system additionally includes personal information regarding the wearer manually imprinted onto the exposed portion of the wearer's skin. The personal information is preferably a telecommunication identifier such as a phone number, ideally a portable cell phone or beeper number of the parent or responsible adult.

### BRIEF DESCRIPTION OF THE DRAWING

The above and related objects, features and advantages of the present invention will be more fully understood by reference to the following detailed description of the presently preferred, albeit illustrative, embodiments of the present invention when taken in conjunction with the appended drawing wherein:

FIG. 1 is a top plan view of a transfer-type tattoo prior to application;

FIG. 2 is a sectional view thereof taken along the line 2-2 of FIG. 1; and

FIG. 3 is a top plan view of the tattoo after transfer thereof onto a wearer's skin and the imprinting of personal information into an exposed skin portion.

### DETAILED DESCRIPTION OF THE PREFERRED EMBODIMENTS

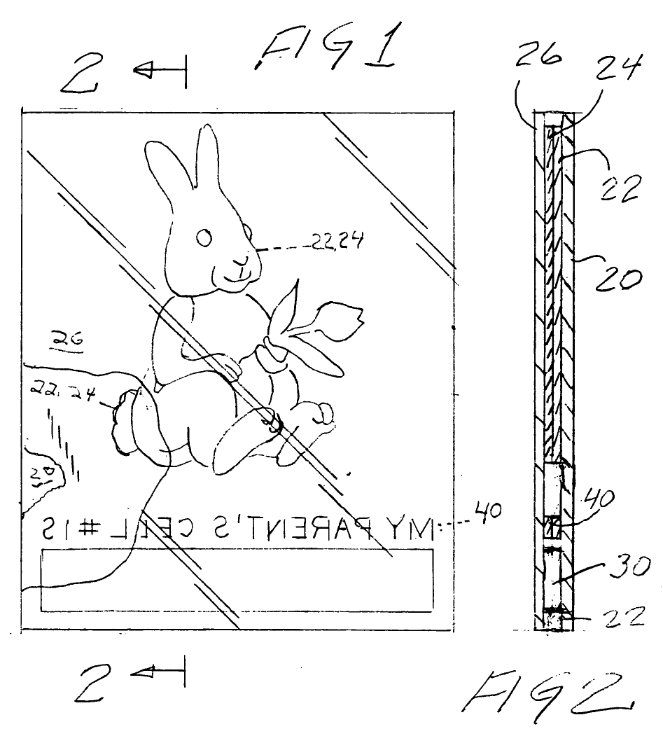

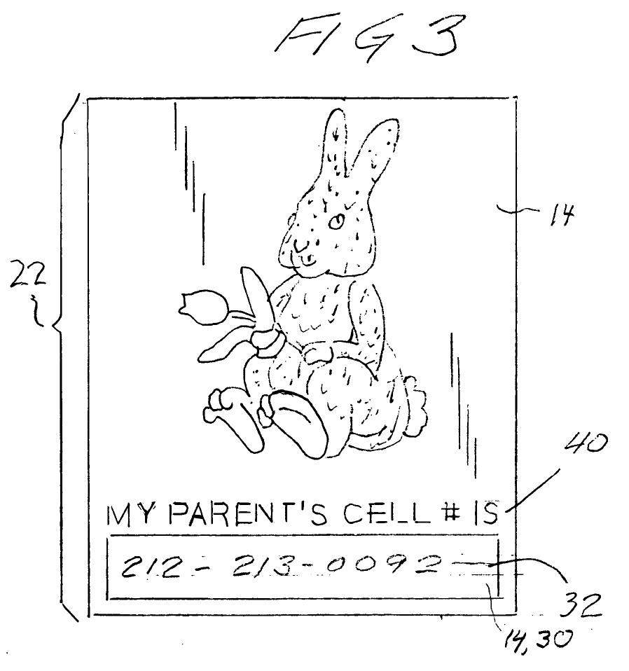

Referring now to the drawing, and in particular to FIGS. 1 and 2 thereof, therein illustrated is a child recovery system according to the present invention, generally designated by the reference numeral 10.

The child recovery system 10 utilizes a transfer-type tattoo, generally designated 12, and may be sold simply as the tattoo 12. The purchaser will supply other elements of the recovery system 10, such as the number of a telecommunication device (e.g., a portable cell phone or beeper) for use in contacting the parent or adult responsible for the wearer-child 14.

The tattoo 12 is of conventional structural design and comprises a laminate, in series, of decal substrate material 20 and a tattoo design 22 releasably disposed over the substrate material 20. The tattoo design 22 is designated to eventually be transferred from the substrate material 20 to the skin of the wearer 14 (see FIG. 3) and hence a pressure-sensitive adhesive 24 is disposed over and in register (i.e., registration) with the tattoo design 22. Initially a removable release sheet 26 is disposed over the adhesive 24. The release sheet 26 is preferably transparent so that the tattoo design 22 can be viewed therethrough (in negative). After manual removal of the release sheet 26 from the remainder of the laminate, the tattoo design 22 is adapted to be transferred from the substrate material 20 to the skin of a wearer 14 (see FIG. 3) due to the effects of the pressure-sensitive adhesive 24 intermediate the tattoo design 22 and the wearer's skin. Release of the tattoo design 22 the substrate material 20 is typically promoted by soaking of the exposed outer surface of the substrate material 20 with water, alcohol, or other solvents which facilitate release of the tattoo design 22 from the substrate material 20.

As described hereinabove, the transfer-type tattoo 12 is conventional in nature and hence need not be described herein in further detail.

Notwithstanding the conventional structure of the tattoo 12, the tattoo design 22 is characterized by two features of the present invention which render it suitable for use in the child recovery system 10 of the present invention.

First, the tattoo design 22 defines therewithin an open area 30 which, after transfer of the tattoo design 22 to the skin of the wearer 14, leaves exposed a portion of the skin of the wearer 14 (within the periphery of the transferred tattoo design) for manual imprinting with personal information 32 regarding the wearer. Accordingly, the transferred tattoo design 22 defines an adhesive-free and ink-free area (i.e., the exposed skin portion) which can be manually imprinted with the personal information 32. Absent such an open area 30 in the tattoo design 22, the ink of the transferred tattoo design 22 (or the adhesive 24 thereunder) would preclude or complicate the manual imprinting of the wearer's skin with the desired personal information 32.

The open area 30 is illustrated as a rectangle but clearly may be of other configurations as well. The dimensions of the open area 30 are sufficiently large to allow manual imprinting therewithin of the desired personal information 32. While only a single open area 30 is shown in the embodiment illustrated, additional open areas may be provided as well, for example, for the receipt of additional personal information 32 or to segment and label the personal information 32 (e.g., a name in one open area 30 and the telecommunication number in another open area 30).

Second, the tattoo design 22 in positive—that is, after transfer to the portion of the skin of the wearer 14—refers a viewer thereof to the personal information 32 imprinted within the open area 30. By way of example, as illustrated in FIG. 3, the transferred tattoo design 22 may include such referring language 40 as “My parent's cell # is ______” and then provide therebelow a box defining the open area 30 to be manually imprinted, e.g., with the cell phone number 32 of a parent.

Clearly the referring language 40 may be modified to reflect the type of personal information 32 being provided. While a child's name and home address may be provided in open areas 30, a telecommunication identifier such as a parent's phone number, and in particular a portable cell phone or beeper number, is preferable as it facilitates inexpensive and immediate communication between the child's parent and any adult stranger who finds the child.

If desired, the tattoos 12 may be made available in a variety of templates having more particularized referring language 40—for example, where the cell # is more particularly identified as belonging to a mother, father, sibling, or the like. Clearly the referring language 40 may be modified to reflect the type of telecommunication intended—e.g., home phone #, work phone #, cell phone #, etc. It may also include a home address, a reward notice, or the like.

Because children generally love tattoos (also sometimes called decals, decalcomanias, or cockamamies) and because the tattoo design 22 may include a character or object which the particular child enjoys associating with (a lovable rose-bearing bunny being indicated in the drawing), the child not only will gladly wear the tattoo design 22 (without attempting to remove it), but will proudly show it to a stranger who finds him. Other suitable subject matter for the tattoo design 22 include automobiles, flowers, animals, clowns, motorcycles and the like.

The tattoo design 22 should be placed on a portion of the skin of the wearer where it will be easily visible by a stranger who encounters the lost child—for example, the back of a hand in winter time and the lower or upper arm during the summer when more of the arm is exposed.

To use the child recovery system 10, the tattoo 12 is applied to a suitable portion of the skin of the wearer in a conventional manner. Thereafter the desired personal information 32 is manually imprinted in the exposed portion of the wearer's skin using as an imprinting element an ordinary pen, crayon, stamp or the like and an imprinting medium (such as ink) which will hopefully remain in place and readable for the relatively brief period of time (typically a few days) during which the tattoo design 22 remains in place and visible.

To summarize, the present invention provides an inexpensive and reliable child recovery system wherein the child-carried component cannot be lost or removed by the child, yet provides the stranger who finds the child with the necessary information to swiftly and easily reunite the child with the responsible adult who lost it. The child will enjoy bearing the child-carried component and may even draw a stranger's attention thereto. The system is simple, inexpensive and easy to manufacture and use.

Now that the preferred embodiments of the present invention have been shown and described in detail, various modifications and improvements thereon will become readily apparent to those skilled in the art. Accordingly, the spirit and scope of the present invention is to be limited only by the appended claims, and not by the foregoing specification.

I claim:

1. In a child recovery system utilizing a transfer-type tattoo comprising a laminate in series of:
(A) a decal substrate material,
(B) a tattoo design, in negative, releasably disposed over said substrate material,
(C) a pressure-sensitive adhesive over and in register with said tattoo design, and
(D) a removable release sheet over said adhesive;
wherein, after removal of said release sheet, said tattoo design is adapted to be transferred from said substrate material to the skin of a wearer;
the improvement wherein:
(i) said tattoo design defines therewithin an open area which after transfer of said tattoo design to the skin of the wearer, exposes a portion of the skin of the wearer for manual imprinting with personal information regarding the wearer, and
(ii) said tattoo design, in positive, refers a viewer thereof to the personal information imprinted within said exposed portion.

2. The system of claim 1 wherein the personal information is a telecommunication identifier.

3. The system of claim 1 wherein the personal information is a phone number.

4. The system of claim 1 wherein the personal information is a portable cell phone or beeper number.

5. A transfer-type tattoo adapted to be transferred to a wearer comprising a tattoo design defining therewithin an open area which exposes a portion of the wearer for manual imprinting with personal information regarding the wearer and referring a viewer thereof to the personal information imprinted within the exposed portion.

6. The system of claim 5 wherein the personal information is a telecommunication identifier.

7. The system of claim 6 wherein the personal information is a phone number.

8. The system of claim 6 wherein the personal information is a portable cell phone or beeper number.
